(19) United States
(12) Patent Application Publication
(10) Pub. No.: US 2022/0020497 A1
(43) Pub. Date: Jan. 20, 2022

(54) BLOOD GLUCOSE DATA SET OPTIMIZATION FOR IMPROVED HYPOGLYCEMIA PREDICTION BASED ON MACHINE LEARNING IMPLEMENTATION INGESTION

(71) Applicant: Novo Nordisk A/S, Bagsvaerd (DK)

(72) Inventor: Anuar Imanbayev, Seattle, WA (US)

(21) Appl. No.: 17/311,003

(22) PCT Filed: Dec. 11, 2019
(86) PCT No.: PCT/EP2019/084634

§ 371 Date: Jun. 4, 2021

Related U.S. Application Data
(60) Provisional application No. 62/779,638, filed on Dec. 14, 2018.

(30) Foreign Application Priority Data
Jan. 25, 2019 (EP) 19153723.2

(51) Int. Cl. G16H 50/30; G16H 50/20; G16H 50/70

(52) U.S. Cl. CPC G16H 50/30; G16H 50/70; G16H 50/20

(57) ABSTRACT

The invention relates to a method for data set expansion for improved hypoglycaemia prediction based on classifier ingestion, and comprises the steps of: providing a raw data set for a subject, the data set comprising a plurality of BG values obtained at a given sampling rate and thereto associated time stamps over a plurality of days N, and performing data transformation by rolling scheme temporal binning of evaluation block values (eHH) as input X to create corresponding prediction values (pHH) as output Y, wherein X is created as a sliding window comprising BG values for a given past period of time T−p, and wherein Y is created as an indicator I indicating whether or not a BG value at a given future time T−f is below a given threshold indicative of a hypoglycaemic condition.

The present disclosure relates generally to systems and methods for assisting patients and health care practitioners in managing insulin treatment to diabetes. In a specific aspect the invention relates to methods for optimized higher data resolutions for machine learning (ML) implementation ingestion.

### BACKGROUND OF THE INVENTION

Diabetes mellitus (DM) is impaired insulin secretion and variable degrees of peripheral insulin resistance leading to hyperglycemia. Type 2 diabetes mellitus is characterized by progressive disruption of normal physiologic insulin secretion. In healthy individuals, basal insulin secretion by pancreatic β cells occurs continuously to maintain steady glucose levels for extended periods between meals. Also in healthy individuals, there is prandial secretion in which insulin is rapidly released in an initial first-phase spike in response to a meal, followed by prolonged insulin secretion that returns to basal levels after 2-3 hours. Years of poorly controlled hyperglycemia can lead to multiple health complications. Diabetes mellitus is one of the major causes of premature morbidity and mortality throughout the world.

Effective control of blood/plasma glucose (BG) can prevent or delay many of these complications but may not reverse them once established. Hence, achieving good glycemic control in efforts to prevent diabetes complications is the primary goal in the treatment of type 1 and type 2 diabetes. In particular, frequent changes in insulin dosage titration are key to helping stabilize blood glucose levels in patients (Bergenstal et al., “Can a Tool that Automates Insulin Titration be a Key to Diabetes Management?” Diabetes Tech. and Thera. 2012; 14(8) 675-682). Smart titrators with adjustable step size and physiological parameter estimation and pre-defined fasting blood glucose target values have been developed to administer insulin medicament treatment regimens. Optimal initiation and titration methods for the long-acting basal insulins are still being determined. However, evidence suggests that many patients often do not receive insulin doses titrated sufficiently to achieve target levels of glucose control (remaining on suboptimal doses and failing to reach treatment targets) (Holman et al., “10-year follow-up of intensive glucose control in type 2 diabetes,” N. Engl. J. Med. 2008; 359: 1577-1589).

One of the major problems with insulin regimens is the lack of patient autonomy and empowerment. Patients often must visit clinics to have new titrations calculated. When a clinic has to titrate the insulin dosages for the patient, there is a natural limitation on the possible frequency of changing the titration dose. Self-titration regimens facilitate empowerment of patients, allowing them to become more involved in their treatment, which can result in improved glycemic control (Khunti et al., “Self-titration of insulin in the management of people with type 2 diabetes: a practical solution to improve management in primary care,” Diabetes, Obes., and Metabol. 2012; 15(8) 690-700). Patients who take an active role in the management of their diabetes and titration of their insulin may feel more empowered to take charge of their selfcare and have a stronger belief that their actions can influence their disease, thus leading to better treatment outcomes (Norris et al., “Self-management education for adults with type 2 diabetes: a meta-analysis on the effect of glycemic control.” Diabetes Care. 2002; 25:1159-71; Kulzer et al., “Effects of self-management training in type 2 diabetes: a randomized, prospective trial,” Diabet. Med. 2007; 24:415-23; Anderson et al., “Patient empowerment: results of a randomized controlled trial.” Diabetes Care. 1995; 18:943-9). Further, when patients have control of their own titration, the frequency of titrations increases, which increases the likelihood that patients will achieve desired blood glucose levels.

However, with a more aggressive titration approach the risk of a hypoglycemic event (“hypo”) will be higher, a risk that is further enhanced in case of a titration regimen based on multiple daily injections (MDI). Correspondingly, a number of solutions for short term hypo prediction (STHP) have been proposed such as Kovatchev et al. (TypeZero & University of Virginia group) “Evaluation of a New Measure of Blood Glucose Variability in Diabetes”, Diabetes Care, Vol 29(11), November 2006, Sparacino et al. (Cobelli Lab in University of Padova) “Glucose Concentration can be Predicted Ahead in Time From Continuous Glucose Monitoring Sensor Time-Series”, IEEE Transactions on Biomedical Engineering, Vol. 54(5) May 2007, Franc et al. (Volunits with Sanofi) “Real-life application and validation of flexible intensive insulin-therapy algorithms in type 1 diabetes patients”, Diabetes Metab. 2009 December, 35(6): 463-8, and Sudharsan et al. (WellDoc)(LTHP 24-hours ahead literature comparison) “Hypoglycemia Prediction Using Machine Learning Models for Patients with Type 2 Diabetes”, Journal of Diabetes Science and Technology 2015, Vol. 9(1) 86-90.

Addressing this issue US 2008/0154513 discloses a method, system, and computer program product related to the maintenance of optimal control of diabetes and is directed to predicting patterns of hypo-glycemia, hyper-glycemia, increased glucose variability, and insufficient or excessive testing for the upcoming period of time, based on blood glucose readings collected by a self-monitoring blood glucose (SMBG) device. The method for identifying and/or predicting patterns of hyper-glycemia of a user comprises the steps of acquiring a plurality of SMBG data points, classifying the SMBG data points within periods of time with predetermined durations, evaluating glucose values in each period of time, and indicating risk of hyper-glycemia for a subsequent period of time based on said evaluation. The evaluation may comprise the steps of determining individual deviations towards hyper-glycemia based on said glucose values, determining a composite probability in each said period of time based on individual and absolute deviations, and comparing said composite probability in each period of time against a pre-set threshold. The periods of time may comprise splitting twenty-four hour days into time bins with predetermined durations.

Addressing the above issues and to better mitigate the risk of hypos it is an object of the present invention to provide methods and systems improving the ability to predict future hypos to dampen a current dose recommendation, this enabling more accurate titration regimens and thereby treatment of type 2 diabetes. It is a specific object of the present invention to provide methods for data set optimization allowing improved hypoglycaemia prediction based on classifier ingestion and machine learning algorithms. Such methods should use a transparent and constrained approach making them better suited to be approved by authorities for use in a dose guidance system.

### DISCLOSURE OF THE INVENTION

In the disclosure of the present invention, embodiments and aspects will be described which will address one or more of the above objects or which will address objects apparent from the below disclosure as well as from the description of exemplary embodiments.

In a first aspect of the present invention a method for data set optimization for improved hypoglycaemia prediction based on classifier ingestion is provided, comprising the steps of: providing a raw data set for a subject, the data set comprising a plurality of BG values obtained at a given sampling rate and thereto associated time stamps over a plurality of days N, performing data transformation by rolling scheme temporal binning of evaluation block values (eHH) as input X to create corresponding prediction values (pHH) as output Y, wherein X is created as a sliding window comprising BG values for a given past period of time T−p, and wherein Y is created as an indicator I indicating whether or not a BG value at a given future time T−f is below a given threshold indicative of a hypoglycaemic condition.

In general, prediction models are only as good as the data that they're trained on. By the above method the same amount of data can be utilized in more efficient and better ways that fit and adapt accordingly to machine learning algorithms, such as the Random Forest (RF) classifier.

In contrast, a previous attempt directed to predicting patterns of hypo-glycemia as disclosed in US 2008/0154513 has relied on simple temporal binning of BG data and subsequent traditional mathematical analysis of the organized data.

Data transformation may be performed for at least two different past periods of time T−p. T−f may correspond to T−p, e.g. a 15 minutes prediction value is based on 15 minutes of BG values.

In an exemplary embodiment the step of data transformation is preceded by the step of performing data expansion by rolling scheme temporal binning of daily BG values into evaluation blocks for M days, M being equal to or larger than 2 and less than the plurality of days N.

Such a data expansion is relevant when a raw data set obtained is based on an M-day insulin titration regimen, e.g. three days with the same insulin dose before a change, such a regimen typically being used for titration of basal insulin as indicated in the Instructions for Use for a given basal insulin. For a data set based on bolus insulin M=1 would be relevant. Indeed, if M=1 no real expansion takes place.

In an exemplary embodiment the step of providing a raw data set is followed by the step of performing data preparation with re-sampling corresponding to a nominal sampling rate and with creation of interpolated BG values to replace missing BG values.

In a further aspect of the present invention a method for training a classifier is provided, comprising the steps of providing a data set optimized as described above, ingesting the optimized data set in a classifier, and train the classifier based on the ingested data set. The classifier may be a Random Forest classifier.

In a further aspect of the present invention a method for predicting a future BG value is provided, comprising the steps of obtaining an evaluation series of BG values from a subject, ingesting the evaluation series of BG values into a classifier having been trained as described above, and providing a predicted BG value. The data set on which the classifier has been trained may have been obtained from the same subject as the evaluation series of BG values. The evaluation series of BG values may be obtained by continuous blood glucose monitoring (CGM), e.g. producing a BG value every 5 minutes.

In a yet further aspect of the present invention a computing system for performing temporal optimization of a dataset from a subject is provided, the computer system comprising one or more processors and a memory, the memory comprising instructions that, when executed by the one or more processors, perform a method as defined above in accordance with the different aspects of the present invention.

In a specific exemplary embodiment data temporal optimization and expansion using the same amount of data but in more expanded, smarter, and fitting ways is provided by performing the following steps:

(1) Missing data handling: 5-minute resampling with spline interpolation solution: data size increases correspondingly with missing data that achieves the data quality processing requirement of data preparation with a piece of software code.

(2) Evaluation Historical Horizon (eHH) with rolling scheme temporal binning: 3-day block binning with temporally optimized rolling daily scheme as opposed to the standard sequential scheme in order to bin a series of CGM measurements nestled within clinically derived interval of 3 days study block, or evaluation historical horizon (eHH) of 3 days back.

(3) Hypoglycemia Prediction Historical Horizon (pHH) with rolling scheme temporal binning: A software program that repeatedly makes a prediction of hypoglycemia at some future interval of time ahead, prediction horizon (PH) of 15, 30, and 60 minutes ahead, based on a corresponding retrospective interval of time back, or prediction historical horizon (pHH) of 15, 30, and 60 minutes back, respectively. Every 5 minutes, with each step, pHH=PH prediction is made, also on a rolling scheme as opposed to a sequential scheme.

Together, these three steps all increase the size and depth of the original unprocessed BG dataset. Thus, the processed dataset transformed with the three step techniques achieves not only a significantly larger size, but also depth and operational ingestibility directly and swiftly into ML classifier formats. An unprocessed or raw dataset cannot be readily or immediately ingested or fed into ML classifier formats with the same efficiency.

Together, the spline missing data interpolation with the rolling scheme temporal bins of evaluation and prediction historical horizon intervals result in optimization of CGM resolution data in order to deliver more accurate predictions of hypoglycemia with high sensitivities (correct prediction of hypoglycemia events) and high specificities (correct prediction of non-hypoglycemia events).

### BRIEF DESCRIPTION OF THE DRAWINGS

In the following embodiments of the invention will be described with reference to the drawings, wherein

FIG. 1 illustrates an example data preparation module in accordance with an embodiment of the present disclosure,

FIG. 2 illustrates an example data transformation module in accordance with an embodiment of the present disclosure,

FIG. 3 illustrates an example pointer lookup table in accordance with an embodiment of the present disclosure,

FIG. 4 illustrates an example temporal bin optimization in accordance with an embodiment of the present disclosure,

FIGS. 5, 6 and 7 illustrate for different pHH values an example hypoglycemia determination module in accordance with an embodiment of the present disclosure,

FIG. 8 illustrates an example saving of training results for subsequent ML processing in accordance with an embodiment of the present disclosure,

FIGS. 9 and 10 illustrate an example Random Forest (RF) Classifier implementation in accordance with an embodiment of the present disclosure,

FIGS. 11 and 12 illustrate RF classifier results in accordance with an embodiment of the present disclosure,

FIGS. 13 and 14 illustrate RF classifier results compared with literature results, and

FIGS. 15-27 collectively illustrate a working example in accordance with an embodiment of the present disclosure.

In the figures like structures are mainly identified by like reference numerals.

### DESCRIPTION OF EXEMPLARY EMBODIMENTS

The present disclosure relies upon the acquisition of sets of training and test data that include information relating to at least one subject. The dataset(s) include at least a plurality of blood glucose measurements of the subject taken over a time course to establish a blood glucose history, and for each respective glucose measurement in the plurality of blood glucose measurements a corresponding glucose timestamp representing when in the time course the respective glucose measurement was made, and one or more basal insulin injection histories, where the injection history includes a plurality of injections during all or a portion of the time course and, for each respective injection in the plurality of injections, a corresponding dose event amount and a dose event timestamp representing when in the time course the respective injection event occurred.

### STHP Classifier: Data Preparation and Data Transformation of STHP Classifier

In order to determine prediction or detection of hypoglycaemia or low blood glucose level adverse event in the short-term, prediction horizon (PH) of 15 minutes up to 60 minutes ahead, then current, experimental, and future machine learning methodologies require optimizations and adaptations to fully ingest, recruit and exploit different temporal resolutions from Self-Monitoring of Blood Glucose (SMBG) at 1 or 2 points per day to flash glucose monitor (FGM) at 15 minute intervals or continuous glucose monitor (CGM) at 5 minute intervals.

In general, prediction models are only as good as the data that they're trained on. Thus, improving data quality or utilizing the same amount of data in more efficiently is of paramount importance and value. This present solution aims at exploiting not just more data with higher temporal resolution of CGM but also utilizing this data in smarter, better ways that fit and adapt accordingly to machine learning algorithms, such as the Random Forest (RF) classifier. For example, in the space of 12 PM-3 PM interval, the SMBG low resolution with Dexcom reporting in hourly intervals, it is only possible to obtain 3 intervals. With CGM high resolution and with full data optimization, 25 intervals are able to be obtained and fed into a ML model such as the Random Forest (RF) classifier.

Current configuration or utilization of CGM data for random forest classifier algorithm is as follows: For example, to predict hypoglycemia in the next 60 minutes (PH=60 minutes ahead), utilize the past 60 minutes as input prediction historical horizon (pHH), yet still constrained within the past 3-day block of the evaluation historical horizon (eHH). Without CGM data, just SMBG data, the temporal shift occurs by each hour.

For example, with SMBG data, in the space of e.g. 3 hours from 12 PM to 3 PM, there are only 3 temporal data intervals: 1) first interval from 12 PM until 1 PM, 2) second interval from 1 PM to 2 PM, and 3) third interval from 2 PM to 3 PM.

This makes sense within the constraints of other measurement schemes such as SMBG or other devices, but not for CGM. This lower resolution scheme fails to optimize and fully exploit the higher resolution data from CGM.

CGM temporal optimization adapts 25 temporal data intervals in the same space of 3 hours, each at 5-minute intervals, as constrained by CGM.

12 PM-3 PM: SMBG low resolution (Dexcom reports in hourly intervals): 3 intervals, CGM high resolution (full optimization): 25 intervals.

So in short, the above is prediction historical horizon (pHH) temporal bin optimization. Thus, with this temporal data optimization and adaptation, instead of having just 3 data intervals prepped for machine learning random forest classifier, 25 data temporal intervals are prepped and ready for machine learning random forest classifier, increasing data availability and training use cases.

While understandably, this full utilization of CGM data may be viewed as just the next logical step, the true improvement lies in applying the higher resolution of CGM data for machine learning algorithms, several of which, for example time series ARIMA models, fare poorly with that many (288 points per day) seasonal parameters to capture daily variation, even when there is obviously a strong seasonal component occurring daily in the data, captured by other functions such as seasonal_decompose from statsmodels package.

Without these CGM data optimization and adaptation methods and functions, then the machine learning algorithms such as the random forest classifier are going to be poorly trained, fitted, and representative of the data that they're trying to make predictions on.

Medical and science rationale for utilizing intermediate 5 minute intervals is that as long as the assumptions of temporal linearity, order, and minimum data quality are maintained, where each 15 minute, 30 minute or 60 minute interval is projected only ahead into the future and follows linearly after each other in 5-minute increments, then it makes no difference whether one applies the 12 PM-1 PM window vs. 12:05 PM-1:05 PM window, except for the new data trends that may be captured in the new window.

For example, within an SMBG resolution of a single point at each hour, instead of CGM resolution of a single point at each 5-minute interval, if say the interval 12 PM-1 PM is missing, there is no way to fill that data in except by extrapolation which is risky. With CGM resolution, if the 12 PM-1 PM interval is missing but the 12:05 PM-1:05 PM is available, then that CGM 5-minute interval shifted hourly duration of 12:05 PM-1:05 PM becomes the accepted data.

With SMBG resolution, if the 1-2 PM interval is missing, then it's possible to interpolate between the 12-1 PM interval and 2-3 PM interval, though it raises some risk but not as much as extrapolation. With SMBG resolution, if the 2-3 PM interval is missing, it's similar situation as 12-1 PM missing, then extrapolation would be needed to fill that missing data in. Basically, the edge cases of the intervals require extrapolation, while in-between cases or intervals of missing data require interpolation. Both are risky, but interpolation is less risky than extrapolation.

CGM data optimization steps remove this need for interpolation and extrapolation by utilizing its higher resolution and being able to resort to other 5-min shifted hourly intervals instead, within of course, medical constraints. For example, if more than 20 minutes is missing, than it's inadvisable to substitute say 12:25 PM-1:25 PM interval (with all intervals between 12 and 12:25 PM missing, basically 5 intervals missing) for the missing 12-1 PM interval. Otherwise, from the medical, scientific, and physiologic perspective, within 20 minutes or 4 5-minute intervals, one can substitute, average, or interpolate between each other, which allows for writing of an adaptation function that can reliably fit or adapt to machine learning algorithms such as random forest classifier, even with missing, incomplete, or corrupted data, as long as some threshold of data quality and linearity is met, which is a far more lenient threshold with the higher temporal data resolution of CGM vs. the very strict and demanding threshold with the lower temporal data resolution of SMBG and other methodologies and devices.

Another way to think about this is the following, in terms of data quality. With the CGM optimization of using every possible (but linearly constrained so), there is more room for missing or corrupted data, and for the machine learning algorithm such as random forest classifier do still have enough data to produce a prediction. With the SMBG of only 3 intervals, even if one interval is missing, then the machine learning algorithm of random forest classifier breaks and cannot give prediction for the next hour.

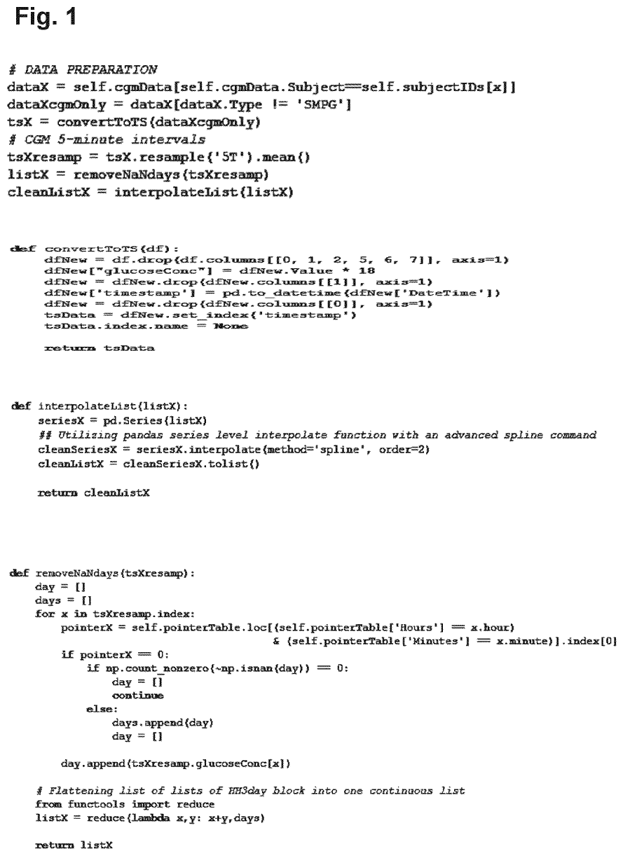

In the following an exemplary embodiment of a data preparation module in Jupyter Notebook code will be described, see FIG. 1.

The Data Preparation module recruits the “convertToTS” and the “removeNaNdays” function. The function “removeNaNdays” itself recruits another function's output lookup table, “pointerTable” to be covered in the Data Transformation module step. Finally the “interpolateList” function is recruited, see FIG. 1.

More specifically, the following takes place:

1. Subject CGM data is read in. Subject CGM data is tabular Data Frame object type.

2. (If there are labels available) subject CGM data removes any “SMPG” or other data labels, leaving only “CGM” data label.

3. Recruiting the “convertToTS” function, Subject CGM data (usually tabular) is converted into a Time Series object for further data preparation.

4. Recruiting Pandas Time Series native resample function with mean, subject CGM Time Series object data that only contains days with at least some CGM data gets further prepped by resampling into “5-T’ or 5-minute bins. If there are no missing data, this step results in the same dataset, but neatly stacked for data analysis. For example, time point of 12:01:43s PM with 85 mg/dL becomes 12:00 PM with the same 85 mg/dL. Also 12:06:21s PM with 92 mg/dL becomes 12:05 PM with the same 92 mg/dL. If there is data missing, then this resampling step is the first substantive increase of the original, raw dataset into a processed, larger dataset with the production of new missing data or NaNs which need to be turned into actual values in subsequent step. Yet first, any full NaN days must be removed. In clinical study, full NaN days are basically the periods in between the baseline and follow-up days. Since both the baseline and follow-up timestamps are in one data object, then the resampling step unfortunately adds needless missing NaN days of non-observation which need to be programmatically removed. This is achieved in the next step.

5. Recruiting the “removeNaNdays” function.

INPUT: subject CGM [Time Series] object data type

PROCESS: scans and removes fully missing NaN days

Rationale: Interpolating entire days between days is also risky. Far less risky is interpolating CGM values within the same day, which will be the next and last step in data preparation.

OUTPUT: subject CGM [List] object data type. No longer [Time Series] object data type!

This function recruits the “pointerTable” function to be explained at Data Transformation module step.

6. Recruiting the “interpolateList” function, this cleaned, processed list of CGM values is finally interpolated with an advanced spline interpolation that fills in any NaN or missing data within days with at least some CGM available.

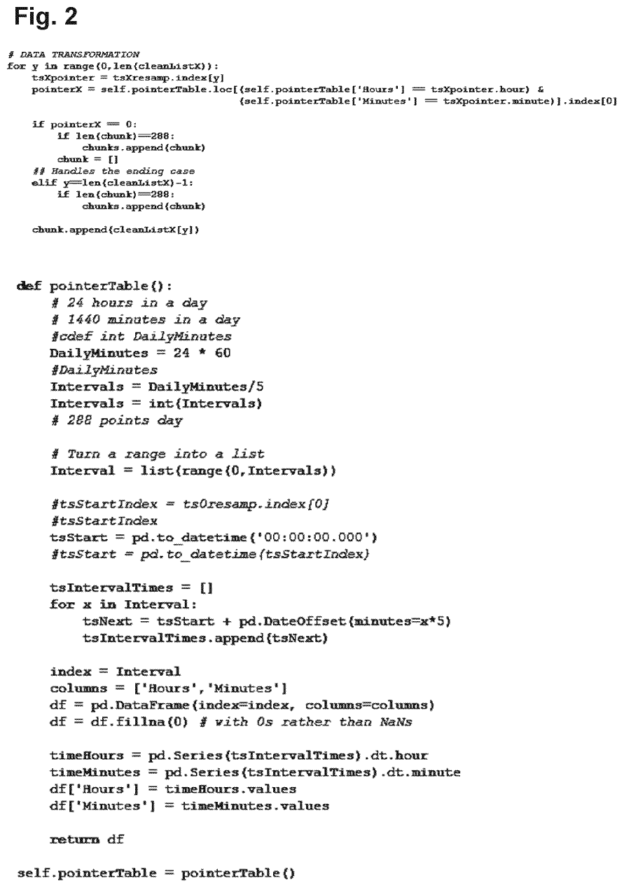

Next, the Data Transformation module recruits the “pointerTable” function's output of a lookup table of a CGM 288 point day, see FIG. 2.

More specifically, the following takes place:

1. “pointerTable” function simply creates once a lookup table of cross-referenced 288 CGM points as IDs.

2. Recruiting the “pointerTable” function, the list of CGMs assigns cross-referenced 288 IDs to align what time point or timestamp in the day that particular value is at.

### CGM Pointer Table Look-Up Sub-Module

From medical & science perspective, it's important to know whether the CGM data point is associated with morning AM or evening PM, especially nocturnal night hours and morning hours, for fasting plasma glucose (FPG) determination and corroboration. A pointer lookup table was devised for a single day in order to still obtain such information without a time series object, just having a list object of CGM value by cross-referencing the 288 IDs of a typical CGM day.

Utilizing the pointer table's 288 IDs of a typical CGM day allows to strip the timestamp component and leave just a list of CGM values. In turn, this list of CGM values can be fed and ingested into ML classifier format algorithms. Unfortunately, a time series object by itself cannot be fed into ML classifier format algorithms. Thus, cross-referencing with a CGM 288-point ID table is necessary.

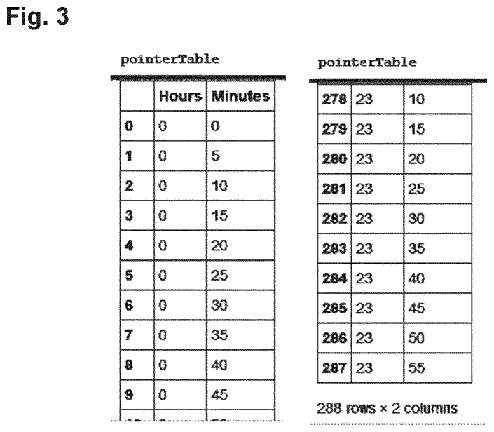

To retain the time-point or hour in the day information (for example id=10 out of 288 CGM points in the day corresponds to time-point of 0:50 AM or 12:50 AM) a CGM 288 Daily 5-minute Steps Pointer Lookup Table is created, see FIG. 3.

For TOP (left graphic), pointer table id=9 corresponds to actual time-point of 12:45 AM, and for BOTTOM (right graphic), pointer table id=287 corresponds to 23:55 PM or 11:55 PM.

Thus, with such a pointer lookup table, it becomes possible to iterate through a list of CGM values (which may contain several days, for example 14-16 days) and understand to what time in the day that CGM value is pointing to, without the time-point data available. Thus, it becomes possible to separate the long list of CGM values into daily chunks, since pointer index of 0 corresponds to a new day, at 12:00 AM.

With the pointer id=0 signifying new or next day, the total list of CGM values can stop populating the previous standalone list for that day and begin a new standalone list of CGM values for the next day. Additionally, the algorithm adds only full days with all 288 points. Any days with less than 288 points do not get added as a full day. In most clinical or realistic trials of users or patients, usually the first and last days or couple of days have less than 288 full points, for example. It is best not to utilize such data since it is difficult to extrapolate, interpolate, or fill in missing data for such corner edge caps of data. Lastly, the algorithm handles the ending case as well, otherwise the last day never gets added appropriately, as confirmed in testing. Outcome result is that the total list of CGM values is now binned into daily chunks or blocks.

Thus, pointerTable gets invoked only in two places in the STHP Classifier codebase:

1. Recruited for the “removeNaNdays” function in order to identify and designate completely missing or NaN days for subsequent removal.

2. Recruited for the Data Transformation module step handling (for loop, if statements) that is mainly tasked with creating evaluation historical horizon (eHH) of 3 day blocks from single day blocks.

INPUT: clean list of CGM values

PROCESS: Cross-referencing with the pointerTable output of “pointerTable” function

OUTPUT: Binning first into daily lists of CGM values (288 points per day or daily chunk)

In the following the Data Optimization module will be described providing CGM Higher Temporal Resolution Optimization by Rolling Scheme Temporal Binning. Adaptation for Ingestion into Machine Learning Random Forest Classifier

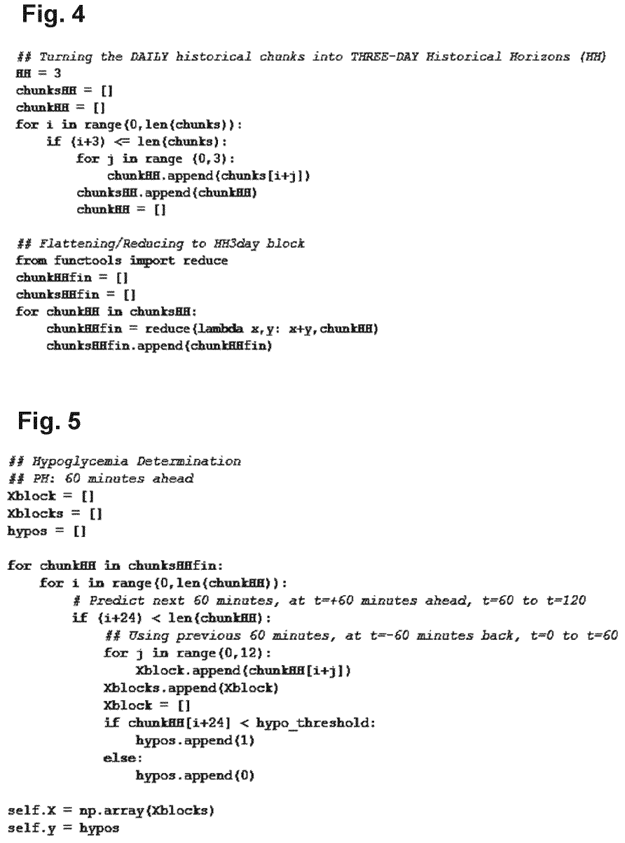

Evaluation Historical Horizon (eHH)—temporal bin optimization, see FIG. 4.

INPUT: Daily lists of CGM values, but un-binned yet into 3-day chunks or blocks

PROCESS: First step utilization of rolling scheme temporal binning

OUTPUT: In turn, these daily chunks can be binned into 3-day chunks or blocks.

Rationale: Binning into daily and 3-day chunks based on Medical & Science considerations and guidelines for patient physiological adjustment period and manageable input consideration for model training period to feed into random forest classifier.

1. The main for loop handles turning DAILY historical chunks into THREE-DAY historical horizon (HH) chunks.

2. Recruiting “reduce” function from “functools” package, the resultant list of lists gets transformed or reduced or flattened into just a single, running list, but this time each list represents not a single day, but 3-days of clinically required observation or evaluation.

Up to this point, CGM data had only one substantive opportunity to grow: at the 5-minute resampling function. All the interpolation function did was to fill in the missing NaNs that the 5-minute resampling step has already grown or expanded. So the interpolation function cannot grow or expand the data. Similarly, the binning into daily chunks is set up in such a way that it just shows how many days there are available in the subject CGM data. No overall data expansion happening in that step. So again, the first substantive opportunity for the dataset to grow was at the 5T or 5-minute resampling step.

However, with this step of binning into 3-day blocks, there is the second substantive opportunity for CGM data to grow and expand.

Typical 3-day block binning within a 12-day available total block: 4 intervals achieved.

| | | |
|---|---|---|
| 1 | 2 | 3 |
| 4 | 5 | 6 |
| 7 | 8 | 9 |
| 10 | 11 | 12 |

The above typical scheme makes sense for SMBG or other device data, where significant recalibrations and calculations must be made between each study block of 3 days. Yet, this makes very little sense for CGM data, which only need calibrations (1-2) within the same day and the calculations can be run daily. Thus, there is no sense to miss the 3-day block from day 2 to day 4, and so on. The medical, scientific, and data science assumptions still hold for the case of this rolling scheme full data optimization with CGM's higher temporal resolution data. These assumptions do not hold for SMBG and other device data and thus the typical scheme is used. Yet, this typical scheme is sub-optimal for CGM implementation, and especially for ML classifier ingestion. Of course, the rolling scheme issues are further resolved and adapted in detail in order to be swiftly recruitable by ML methods from Random Forest (RF) to Support Vector Machine (SVM) to K-Nearest Neighbours (KNN).

Correspondingly, the below optimized and more data gleaning way to bin 3-day blocks is provided.

Optimized 3-day block binning within a 12-day available total block:

| | | |
|---|---|---|
| 1 | 2 | 3 |
| 2 | 3 | 4 |
| 3 | 4 | 5 |
| 4 | 5 | 6 |
| 5 | 6 | 7 |
| 6 | 7 | 8 |
| 7 | 8 | 9 |
| 8 | 9 | 10 |
| 9 | 10 | 11 |
| 10 | 11 | 12 |

10 intervals achieved with this optimized scheme. Basically, total n−3, inclusive.

Hypoglycemia Prediction Historical Horizon (pHH) temporal bin optimization:

INPUT: 3-day chunks or blocks of evaluation Historical Horizon (eHH).

Rationale: This setup avoids temporal pitfalls and errors of bleeding into the next clinical evaluation period of 3 days. Neatly packed for ML analysis.

PROCESS: Second step utilization of rolling scheme temporal binning.

OUTPUT: Prediction Historical Horizon (pHH) is nestled within the evaluation HH (eHH) of 3-day chunks or blocks. This is crucial to setup borders and boundaries that would delineate for machine learning (ML) and also adhere to patient physiological adjustment or alignment. With this second innovative step, this is the third substantive opportunity for input data to grow. Thus, the original, raw input data has been grown or expanded in three substantive steps into the processed and cleaned input data that is now ready for ML classifier format ingestion, model creation, training, and testing.

For pHH=PH=60 minutes, see FIG. 5.

With this last data optimization step of crafting these prediction historical horizons (pHH) for ML classifier input separate and modular from data preparation, transformation, and data adaptation with creation of evaluation historical horizons (eHH), only this hypoglycemia determination changes between different implementations, from pHH=PH=15 to pHH=PH=30 minutes to pHH=PH=60 minutes.

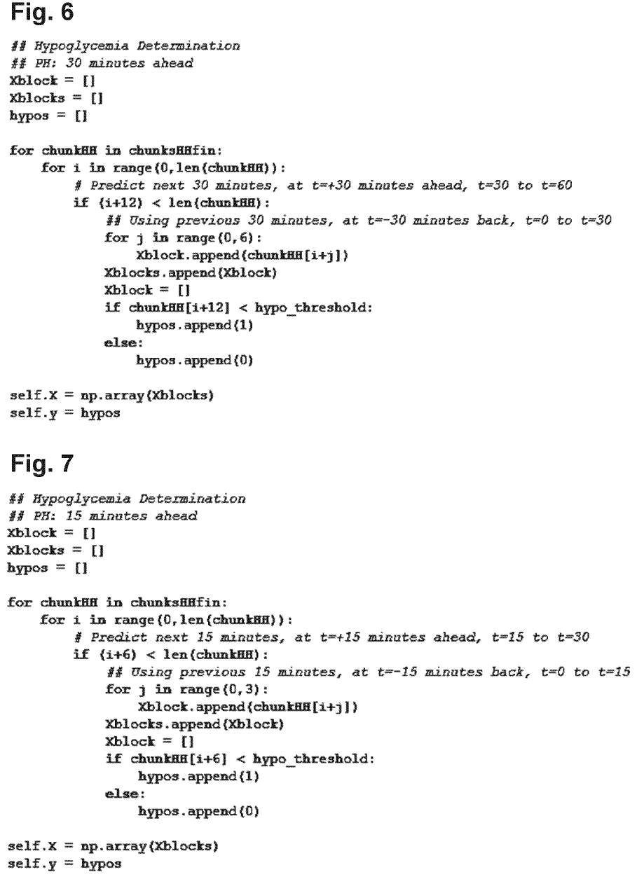

For pHH=PH=30 minutes, see FIG. 6, and for pHH=PH=15 minutes, see FIG. 7.

Up until the now, the exemplary embodiment covered the Compute calculations behind transforming raw, unprocessed CGM data into cleaned, processed, and ML-ingestible data that has been thrice expanded and temporally optimized, and thus can be fed into a Random Forest (RF) classifier model.

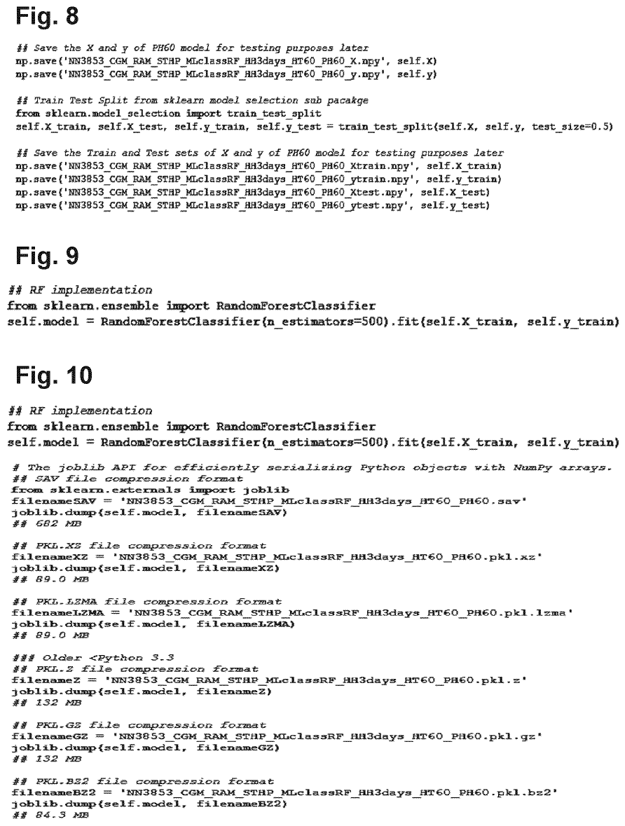

In the Finalized data section focusing on the production and saving of Train-Test X-y sets (see FIG. 8) both the independent variable (the Xs) and the dependent variable (the ys) along with the train-test split dataset portions are saved. These finalized datasets for this particular pHH=PH=60 minutes are then validated in the Test code section, see below.

After these finalized datasets are saved the actual STHP RF Classifier model can be run and crafted with that finalized data input.

### Simple Numeric Example

In the following a simple numeric example will be used to illustrate the above-described data processing steps. The values are generated randomly for this purpose and not based on real data. [KEY] numerator: # day: 12 CGM values per day at # mg/dL. Only pHH of 15-minutes and 30-minutes ahead are possible within this simplified illustrative example of 12 CGM points. In the following calculations are mainly made for pHH of 15 minutes.

0: 1^{st} day: [158, 335, 146, 371, 104, 170, 109, 290, 127, 151, 231, 376]

1: 2^{nd} day: [342, 201, 174, 100, 253, 36, 134, 270, 225, 117, 202, 356]

2: 3^{rd} day: [240, 172, 320, 174, 57, 215, 225, 163, 246, 235, 159, 36]

3: 4^{th} day: [248, 342, 52, 388, 309, 219, 243, 275, 166, 107, 191, 288]

4: 5^{th} day: [279, 74, 146, 276, 284, 334, 201, 185, 187, 151, 242, 114]

5: 6^{th} day: [215, 289, 338, 282, 331, 282, 21, 152, 270, 83, 57, 114]

| E3HH | STEP 10: Triply | STEP 11: Doubly |
|---|---|---|
| BLOCKS | Nested List | Nested List |
| Block 1 | [day #1: 12], | [day #1-3: 36] |
| | [day #2: 12], | |
| | [day #3: 12] | |
| Block 2 | [day #2: 12], | [day #2-4: 36] |
| | [day #3: 12], | |
| | [day #4: 12] | |
| Block 3 | [day #3: 12], | [day #3-5: 36] |
| | [day #4: 12], | |
| | [day #5: 12] | |
| Block 4 | [day #4: 12], | [day #4-6: 36] |
| | [day #5: 12], | |
| | [day #6: 12] | |

pHH=PH=15 SLIDING window of 6.

INPUT: eHH of BLOCK 1:

0: 1^{st} day: [158, 335, 146, 371, 104, 170, 109, 290, 127, 151, 231, 376]

Sliding_Window1=[158, 335, 146, 371, 104, 170]

X1=[158, 335, 146] ˜corresponds to last 3 CGM points of last 15 minutes back

Y1=0˜170>70=0, corresponds to No-Hypo because 170 mg/dL>hypo threshold of 70 mg/dL

So, then X1 would be added or appended to the Xs (or inputs, past CGM BG values) and the Y1 would be added or appended to the Ys (outputs, hypos/non-hypos binary classifier, on/off).

Sliding_Window2=[335, 146, 371, 104, 170, 109]

X2=[335, 146, 371] ˜corresponds to last 3 CGM points of last 15 minutes back

Y2=0˜109>70=0, corresponds to No-Hypo because 109 mg/dL>hypo threshold of 70 mg/dL

Xs and Ys, so far:

Xs=[[158, 335, 146], ˜Xs[0]
[335, 146, 371]] ˜Xs[1]

Ys=[0, 0] ˜Ys[0], Ys[1]

Sliding_Window3=[146, 371, 104, 170, 109, 290]

X3=[146, 371, 104] ˜corresponds to last 3 CGM points of last 15 minutes back Y3=0˜290>70=0, corresponds to No-Hypo because 290 mg/dL>hypo threshold of 70 mg/dL

Xs and Ys, so far:

Xs=[[158, 335, 146], ˜Xs[0]
[335, 146, 371], ˜Xs[1]
[146, 371, 104]] ˜Xs[2]

Ys=[0, 0, 0] ˜Ys[0], Ys[1], Ys[2]

Sliding_Window4=[371, 104, 170, 109, 290, 127]

X4=[371, 104, 170] ˜corresponds to last 3 CGM points of last 15 minutes back

Y4=0˜127>70=0, corresponds to No-Hypo because 127 mg/dL>hypo threshold of 70 mg/dL

Xs and Ys, so far:

Xs=[[158, 335, 146], ˜Xs[0]
[335, 146, 371], ˜Xs[1]
[146, 371, 104], ˜Xs[2]
[371, 104, 170]] ˜Xs[3]

Ys=[0, 0, 0, 0] ˜Ys[0], Ys[1], Ys[2], Ys[3]

Sliding_Window5=[104, 170, 109, 290, 127, 151]

X5=[104, 170, 109] ˜corresponds to last 3 CGM points of last 15 minutes back

Y5=0˜151>70=0, corresponds to No-Hypo because 151 mg/dL>hypo threshold of 70 mg/dL

Xs and Ys, so far:

Xs=[[158, 335, 146], ˜Xs[0]
[335, 146, 371], ˜Xs[1]
[146, 371, 104], ˜Xs[2]
[371, 104, 170], ˜Xs[3]
[104, 170, 109]] ˜Xs[4]

Ys=[0, 0, 0, 0, 0] ˜Ys[0], Ys[1], Ys[2], Ys[3], Ys[4]

Sliding_Window6=[170, 109, 290, 127, 151, 231]

X6=[170, 109, 290] ˜corresponds to last 3 CGM points of last 15 minutes back

Y6=0˜231>70=0, corresponds to No-Hypo because 231 mg/dL>hypo threshold of 70 mg/dL

Xs and Ys, so far:

Xs=[[158, 335, 146], ˜Xs[0]
[335, 146, 371], ˜Xs[1]
[146, 371, 104], ˜Xs[2]
[371, 104, 170], ˜Xs[3]
[104, 170, 109], ˜Xs[4]
[170, 109, 290]] ˜Xs[5]

Ys=[0, 0, 0, 0, 0, 0] ˜Ys[0], Ys[1], Ys[2], Ys[3], Ys[4], Ys[5]

Sliding_Window7=[109, 290, 127, 151, 231, 376]

X7=[109, 290, 127] ˜corresponds to last 3 CGM points of last 15 minutes back

Y7=0˜376>70=0, corresponds to No-Hypo because 376 mg/dL>hypo threshold of 70 mg/dL

Xs and Ys, so far:

Xs=[[158, 335, 146], ˜Xs[0]
[335, 146, 371], ˜Xs[1]
[146, 371, 104], ˜Xs[2]
[371, 104, 170], ˜Xs[3]
[104, 170, 109], ˜Xs[4]
[170, 109, 290], ˜Xs[5]
[109, 290, 127]] ˜Xs[6]

Ys=[0, 0, 0, 0, 0, 0, 0] ˜Ys[0], Ys[1], Ys[2], Ys[3], Ys[4], Ys[5], Ys[6]

In short, just for eHH day #1 of BLOCK 1, 7 pHH=PH=15 Xs (inputs) with corresponding Ys (outputs) were created.

For the rest of the days of eHH BLOCK 1 the values are calculated in the same way.

1: 2^{nd} day: [342, 201, 174, 100, 253, 36, 134, 270, 225, 117, 202, 356]

2: 3^{rd} day: [240, 172, 320, 174, 57, 215, 225, 163, 246, 235, 159, 36]

In the following examples are shown illustrating calculations resulting in the finding of Hypos.

pHH=PH=15

1: 2^{nd} day: [342, 201, 174, 100, 253, 36, 134, 270, 225, 117, 202, 356]

Day2_Sliding_Window1=[342, 201, 174, 100, 253, 36]

Day2_X1=[342, 201, 174] ˜corresponds to last 3 CGM points of last 15 minutes back

Day2_Y1=1˜36<70=1, corresponds to Hypo because 36 mg/dL>hypo threshold of 70 mg/dL

Xs and Ys, so far:

Xs=[[342, 201, 174]]

Ys=[1]

pHH=PH=30

2: 3^{rd} day: [240, 172, 320, 174, 57, 215, 225, 163, 246, 235, 159, 36]

Day3_Sliding_Window1=[240, 172, 320, 174, 57, 215, 225, 163, 246, 235, 159, 36]

Day3_X1=[240, 172, 320, 174, 57, 215] ˜corresponds to last 3 CGM points of last 15 minutes back

Day3_Y1=1˜36<70=1, corresponds to Hypo because 36 mg/dL>hypo threshold of 70 mg/dL

Xs and Ys, so far:

Xs=[[240, 172, 320, 174, 57, 215]]

Ys=[1]

### Random Forest (RF) Classifier Implementation, See FIG. 9.

500 Decision Trees (n_estimators parameter) to be run for random forest classifier is a demanding requirement. Most run at 100 to 300 decision trees. In order to bring the performance and competitiveness of the more simpler but easier to explain decision tree-based Random Forest (RF) classifier against the most cutting-edge, complex but harder to explain neural networks (ANN, CNN, etc.) of hypo prediction algorithms of competitors such as WellDoc, UVA, and others, it was deemed reasonable to bring up the number of decision trees up to 500 from the more standard 100 or 300. Further research & development on tolerance testing and avoiding Out-of-Memory issues on local machines and local host servers and moving into distributed, parallelized computing with Hadoop, MapReduce, and Spark on Amazon Web Services and other such services needs to happen to further fine-tune this parameter of number of decision trees to train and other such parameters.

Data needs to be sufficiently robust to accommodate a high parameter like that. Raw data simply fed will not be able to run with a random forest classifier with that many decision trees. Thus, the innovative data preparation, transformation, adaptation, and especially optimization steps with rolling scheme temporal binning into evaluation and prediction historical horizons (eHH, pHH) were crucially and vitally needed for this classification solution to an otherwise more regression-warranted (but also more regression poor data quality-prone) solution. The classification-based solution is much more robust and resistant against poor data quality, largely thanks to the data expansion and temporal optimization introduced in this invention disclosure.

As shown in FIG. 10, the resultant model may also be saved in joblib API formats that are efficient for serializing Python objects with NumPy arrays, testing different compression formats. The XZ, LZMA, and especially BZ2 formats consistently perform better (smaller size in MB) compression than the Z, GZ, and the especially sub-optimal SAV compression formats.

Summarizing the above disclosure, use of “rolling scheme temporal binning” allows utilizing the same amount of past historical or retrospective data in more expanded, better, smarter, and more fitting ways, effectively growing and increasing the original raw, unprocessed dataset.

Especially with the evaluation and prediction historical horizons (eHH, pHH) constructed with the step of “rolling scheme temporal binning”, the already expanded dataset is further maximized and primed in order to feed even more available data intervals that are transformed and ingestible into ML classification methods such as the Random Forest (RF), Support Vector Machine (SVM), and K-Nearest Neighbours (KNN).

For the LTHP PH=1-day (24 hours), RF achieved 91% accuracy, 90.9% sensitivity, and 91.9% specificity, however, the SVM and KNN performance were poorer. For the LTHP PH=1-day (24 hours), the SVM performed was worse at 86% accuracy, 71.4% sensitivity, and 77.4% specificity. For the LTHP PH=1-day (24 hours), KNN performed was worse at 86% accuracy, 73.2% sensitivity, and 81.7% specificity. Raw CGM data was provided from Novo Nordisk clinical trial NN1218-3853.

Based on these LTHP results, only RF implementation for STHP was implemented in this example (in the figures named “Lombardi”) for a STHP ML Classifier solution. For pHH=PH=30 minutes, the RF implementation of STHP achieved 98% accuracy, 93.59% sensitivity, and 99.75% specificity.

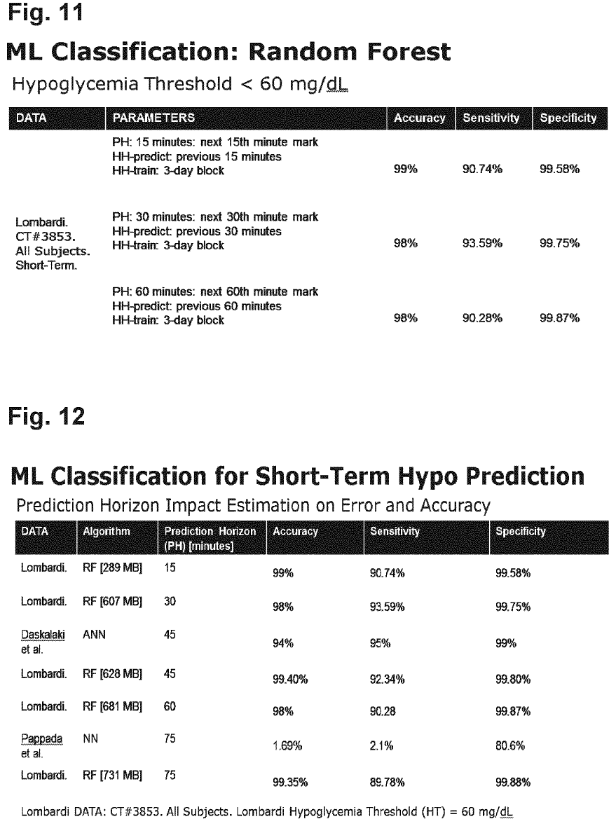

The STHP RF Results for PH15, PH30, PH60 are shown in FIG. 11. In FIG. 12 STHP RF Classifier Results for PH15, PH30, PH45, PH60, PH75 are shown and compared with literature results published by:

Daskalaki et al. “Real-Time Adaptive Models for the Personalized Prediction of Glycemic Profile in Type 1 Diabetes Patients.” Diabetes Technology & Therapeutics Vol. 14(2) 2012. Rationale: From academic literature, Daskalaki et al. paper was used as comparison for the Short-Term Hypoglycemia Predictor (STHP) Classifier Prediction Horizon (PH) at 30 and 45 minutes.

Pappada et al. “Neural Network-Based Real-Time Prediction of Glucose in Patients with Insulin-Dependent Diabetes.” Diabetes Technology & Therapeutics Vol. 13(2) 2011. Rationale: From academic literature, Daskalaki et al. paper was used as comparison for the Short-Term Hypoglycemia Predictor (STHP) Classifier Prediction Horizon (PH) at 75 minutes.

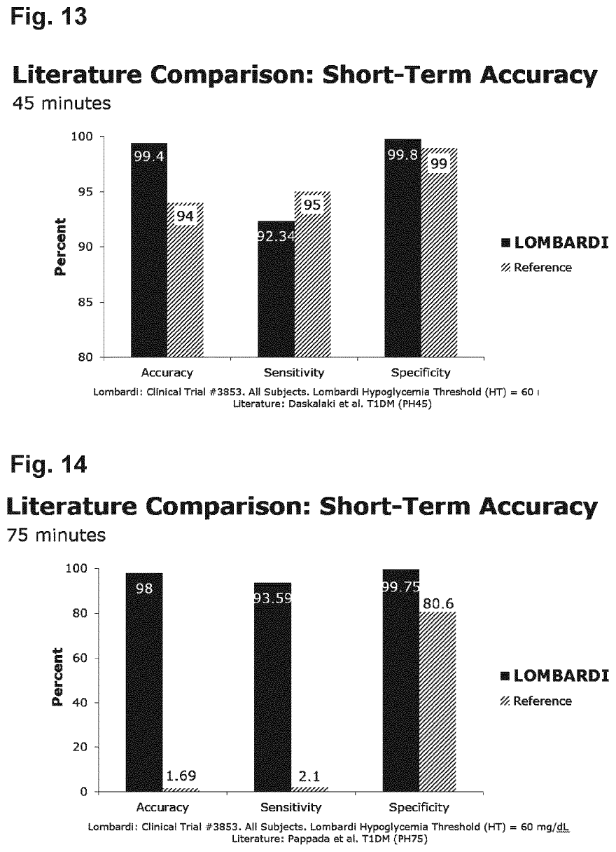

In FIGS. 13 and 14 STHP RF Classifier Results for PH45 respectively PH75 are compared with literature results. As appears, accuracies, sensitivities, and specificities at all prediction horizons of 15, 30, 45, 60, and 75 minutes were achieved that are competitive or even better than literature comparisons from industry and academic sources.

### Working Example

Next a working example (WE) for pHH=PH=60 minutes or STHP RF Classifier 60 minutes will be described, the example covering the test code that achieved the above-referred competitive results by loading the following five files for specific testing and validation purposes:

1. The STHP RF Classifier model file itself: “_PH60.pkl.bz2” suffix

2. Finalized Data of the Test subset of independent variables, Xs: “_Xtest.npy” suffix

3. Finalized Data of the Test subset of dependent variables, ys: “_ytest.npy” suffix

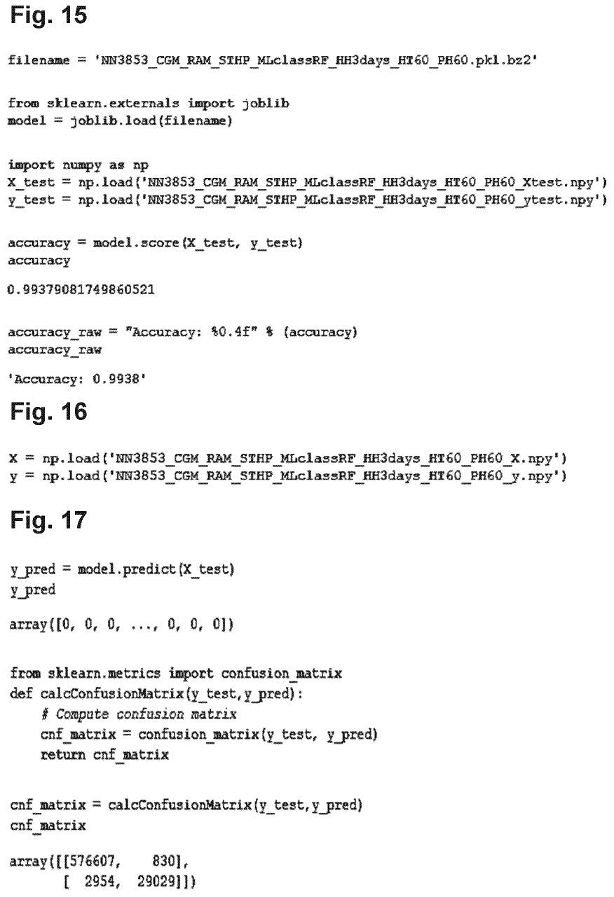

With just the three above file inputs, the following validation test metrics can be computed: raw accuracy, confusion matrix calculations such as sensitivity and specificity as well as the confusion matrix graphic itself, and classification report. See FIG. 15.

4. Finalized Data of ALL independent variables: “_X.npy” suffix

5. Finalized Data of ALL dependent variables: “_y.npy” suffix

These two are only needed for the calculation of cross-validated accuracy. See FIG. 16.

With all these combined, a summary report can be provided for Finalized Data Inputs #1-3:

Validation Test Metrics Results of WE: PH=60 min.

Confusion Matrix Table, see FIG. 17

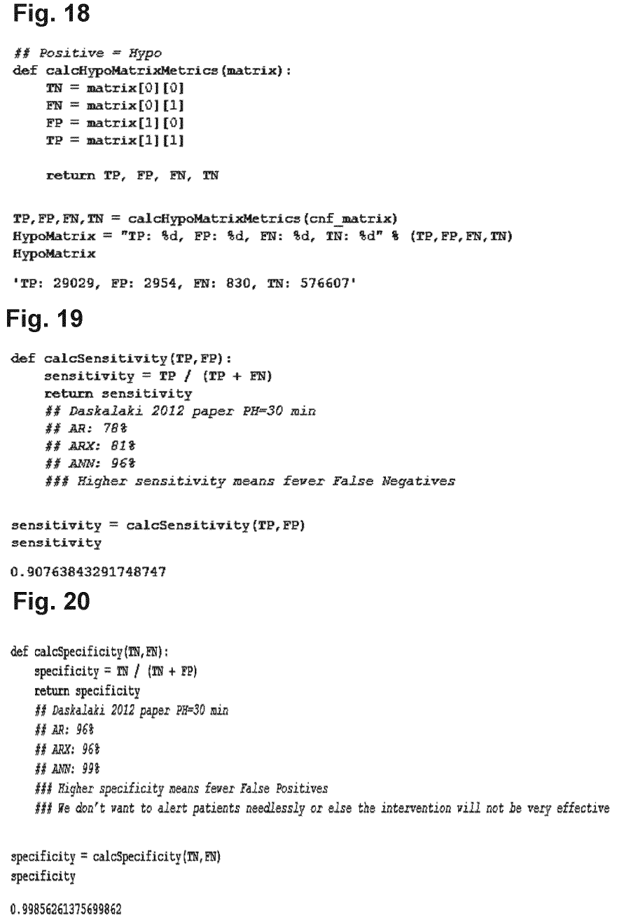

Confusion Matrix Table Calculations: TN, FN, FP, TP, see FIG. 18.

Confusion Matrix Table Calculations: Sensitivity, see FIG. 19.

Confusion Matrix Table Calculations: Specificity, see FIG. 20

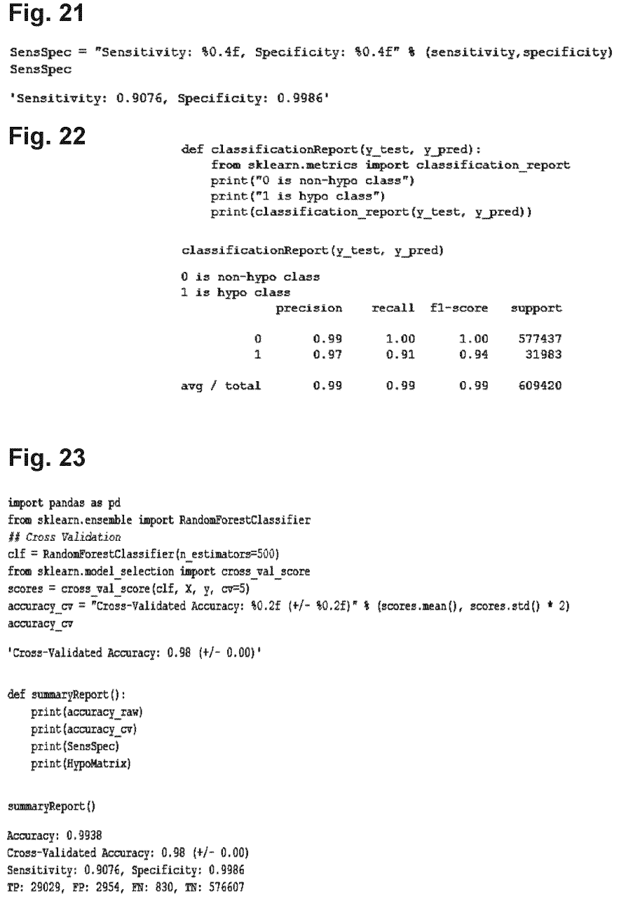

Confusion Matrix Table Calculations: Sensitivity, Specificity string report output, see FIG. 21

Classification Report: Precision, Recall, F1-score, and Support, see FIG. 22.

For Finalized Data Inputs #4-5: Validation Test Metrics Results of WE: PH=60 min: Summary Report: Accuracy, Cross-Validated Accuracy, Sensitivity, Specificity, Hypo Matrix (TN, FN, TP, FP), see FIG. 23.

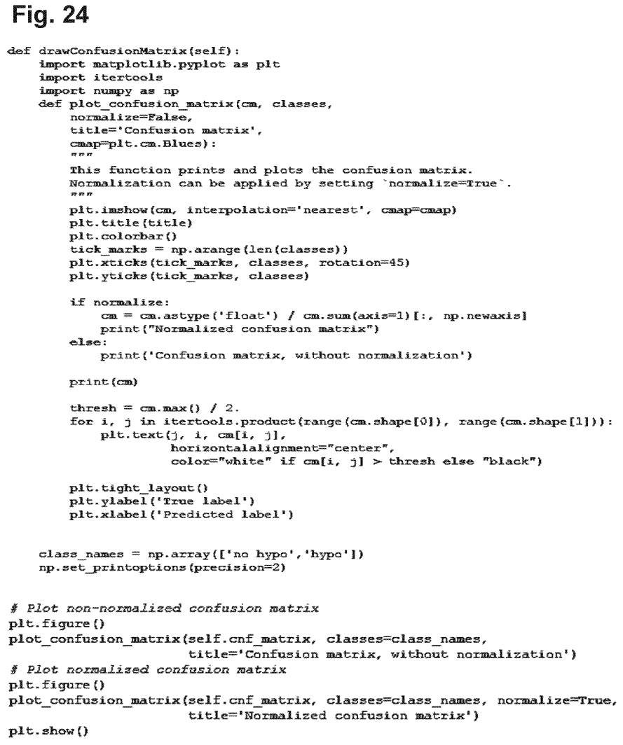

Confusion Matrix Function, see FIG. 24.

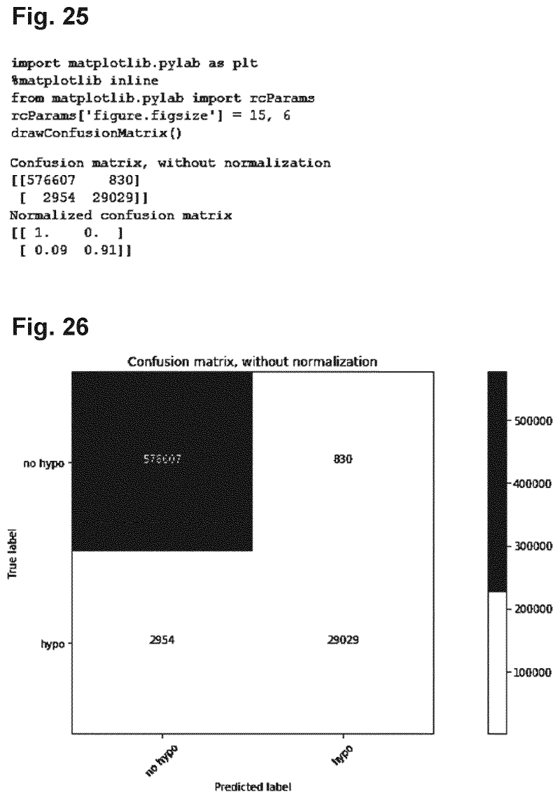

Confusion Matrix Function: Output (1/3), see FIG. 25.

Confusion Matrix Function: Output (2/3): Without normalization, see FIG. 26.

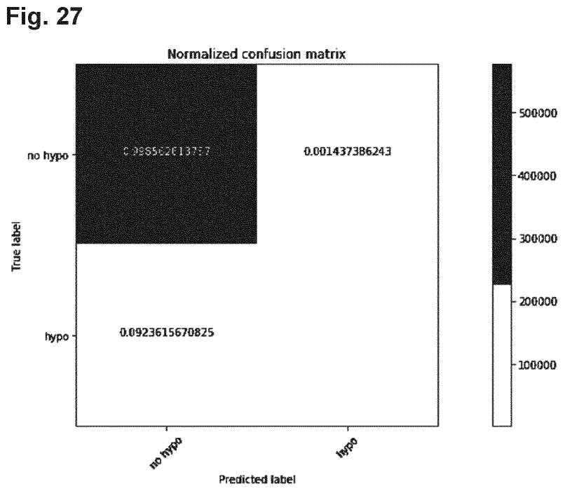

Confusion Matrix Function: Output (3/3): With normalization, see FIG. 27.

### REFERENCES CITED AND ALTERNATIVE EMBODIMENTS

All references cited herein are incorporated herein by reference in their entirety and for all purposes to the same extent as if each individual publication or patent or patent application was specifically and individually indicated to be incorporated by reference in its entirety for all purposes.

All headings and sub-headings are used herein for convenience only and should not be construed as limiting the invention in any way.

The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

The citation and incorporation of patent documents herein is done for convenience only and does not reflect any view of the validity, patentability, and/or enforceability of such patent documents.

The present invention can be implemented as a computer program product that comprises a computer program mechanism embedded in a non-transitory computer readable storage medium. For instance, the computer program product could contain the program modules shown in any combination of FIGS. 1 and 2 and/or described in FIG. 4. These program modules can be stored on a CD-ROM, DVD, magnetic disk storage product, USB key, or any other non-transitory computer readable data or program storage product.

Many modifications and variations of this invention can be made without departing from its spirit and scope, as will be apparent to those skilled in the art. The specific embodiments described herein are offered by way of example only. The embodiments were chosen and described in order to best explain the principles of the invention and its practical applications, to thereby enable others skilled in the art to best utilize the invention and various embodiments with various modifications as are suited to the particular use contemplated. The invention is to be limited only by the terms of the appended claims, along with the full scope of equivalents to which such claims are entitled.

1. A method for data set optimization for improved hypoglycaemia prediction based on classifier ingestion, comprising the steps of:
providing a raw data set for a subject, the data set comprising a plurality of BG values obtained at a given sampling rate and thereto associated time stamps over a plurality of days N,
performing data transformation by rolling scheme temporal binning of evaluation block values (eHH) as input X to create corresponding prediction values (pHH) as output Y,
wherein X is created as a sliding window comprising BG values for a given past period of time T−p, and
wherein Y is created as an indicator I indicating whether or not a BG value at a given future time T−f is below a given threshold indicative of a hypoglycaemic condition.

2. A method for data set optimization as in claim 1, wherein the step of data transformation is preceded by the step of:
performing data expansion by rolling scheme temporal binning of daily BG values into evaluation blocks for M days, M≥2, M<N.

3. A method for data set optimization as in claim 2, wherein the raw data set obtained is based on an M-day insulin titration regimen.

4. A method for data set optimization as in claim 1, wherein the step of providing a raw data set is followed by the step of:
performing data preparation with re-sampling corresponding to a nominal sampling rate and with creation of interpolated BG values to replace missing BG values.

5. A method for data set optimization as in claim 1, wherein data transformation is performed for at least two different past periods of time T−p.

6. A method for data set optimization as in claim 5, wherein T−f corresponds to T−p.

7. A method for training a classifier, comprising the steps of:
providing a data set optimized as defined in claim 1,
ingesting the optimized data set in a classifier, and
train the classifier based on the ingested data set.

8. A method for training a classifier as in claim 7, wherein the classifier is a Random Forest classifier.

9. A method for predicting a future BG value, comprising the steps of:
obtaining an evaluation series of BG values from a subject,
ingesting the evaluation series of BG values into a classifier having been trained as defined in claim 7, and
providing a predicted BG value.

10. A method for predicting a future BG value as in claim 9, wherein the evaluation series of BG values is obtained by continuous blood glucose monitoring (CGM).

11. A computing system for performing temporal optimization of a dataset from a subject, wherein the computer system comprises one or more processors and a memory, the memory comprising:
instructions that, when executed by the one or more processors, perform a method as defined in claim 1.
